(19) United States
(12) Patent Application Publication
(10) Pub. No.: US 2012/0253317 A1
(43) Pub. Date: Oct. 4, 2012

(54) ADIPOSE TISSUE MANAGEMENT SYSTEMS

(75) Inventor: Mel Bircoll, Los Angeles, CA (US)

(73) Assignee: Birscet, LLC, Encinitas, CA (US)

(21) Appl. No.: 13/516,196

(22) PCT Filed: Dec. 17, 2010
(86) PCT No.: PCT/US10/61149

§ 371 Date: Jun. 14, 2012

Related U.S. Application Data
(60) Provisional application No. 61/288,114, filed on Dec. 18, 2009.
(60) Provisional application No. 61/288,091, filed on Dec. 18, 2009.

(51) Int. Cl. A61M 37/00; C12N 5/077; A01N 1/02

(52) U.S. Cl. 604/506; 435/374; 435/325

(57) ABSTRACT

The utilization of stored fat cells is provided. Fat cells removed from a patient may be subdivided into a number of fat cell subdivisions for utilization in a series of medical procedures. The fat cell subdivisions can be prepared for short term storage for use in medical procedures to be performed within a defined period time or long term storage for use in one or more medical procedures to be performed in the future. Medical practitioners can utilize stored fat cell subdivisions in a series of procedures on a patient.

### CROSS-REFERENCE TO RELATED APPLICATIONS

This application claims the benefit of U.S. Provisional Application No. 61/288,114, filed Dec. 18, 2009, and U.S. Provisional Application No. 61/288,091 filed Dec. 18, 2009, the entirety of these applications are hereby incorporated by reference herein.

### BACKGROUND

As early as the late 1800's, the method of transferring adipose tissue, also known as fat, from one part of a person's body to another has been attempted. Not until more recent years has the procedure of transferring fat gained popularity due to improved methods of injecting the fat cells. The concept of transferring fat to enhance parts of the body has several favorable attributes such as being a natural alternative as opposed to synthetic (e.g. silicone, saline), and for having a more natural look and feel than synthetic implants. One significant example is breast augmentation which is performed on over 400,000 thousand women a year. Currently, women are able to choose different types of synthetic implants (saline, silicone) and it is acknowledged that other synthetic options will be available in the future (e.g. hydrogel, FDA approved gels). Saline implants provide a safer alternative to silicone since it is still unclear as to the effects of implanting silicone. However, silicone implants provide a significantly more natural feel and appearance than saline implants. There are also risks associated with breakage of either of the silicone or saline implants as well as the potential for any synthetic implant to be rejected by the body.

More recently, human adipose tissue has been identified as a readily available source of adult stem cells. These multi-potent cells are capable of differentiating into adipocytes, osteoblasts, and chondrocytes, and are believed to have therapeutic potential for a wide variety of disease states and conditions.

Notwithstanding the foregoing, there remains a need for devices and systems for managing human adipose tissue from the point of extraction through various end uses such as near term and future tissue augmentation, as well as preservation for future therapeutic use.

### SUMMARY

There is provided in accordance with one aspect of the present invention, a method of managing adipose tissue. The method comprises the steps of performing liposuction on a patient, to harvest a volume of adipose tissue. The volume of tissue is divided into at least two containers, and at least one of the containers is cryopreserved.

A first container may be provided with a first volume of adipose tissue and a second container may be provided with a second, different volume of adipose tissue. In some implementations of the invention, a first set of at least two containers each has a first volume, and a second set of at least two containers has a second volume.

The method may additionally comprise the step of placing at least the first container into a removable sterile enclosure prior to the cryopreserving step. The method may additionally comprise the step of associating patient identifying information with each container.

At least a first container may be stored at a first location such as the physician's office, for reinjection into the patient for cosmetic purposes. At least a second container may be sent to a long term cryogenic storage facility.

In accordance with another aspect of the present invention, there is provided a method of performing a cosmetic procedure on a patient. The method comprises the steps of harvesting adipose tissue from the patient, and processing the adipose tissue. The processed adipose tissue is divided into at least two sets. At least a portion of a first set is injected back into the patient, and the second set is sent to a long term cryogenic storage facility.

In accordance with a further aspect of the present invention, there is provided a method for managing tissue removal. The method comprises the steps of determining a number of fat cell subdivisions for storing fat cells removed from a patient, and designating a storage designation for the number of fat cells subdivisions, wherein the designated storage designation corresponds to at least one of long term storage and short term storage. A designation of a procedure utilizing one or more stored fat cell subdivisions is obtained, and a number of fat cell subdivisions to be utilized in the procedure is determined. Preparation of previously stored fat cell subdivisions based upon the number of determined fat cell subdivisions is caused, where in the previously stored fat cell subdivisions may be fat cell subdivisions stored in at least one of short term storage and long term storage.

Further features and advantages of the present invention will become apparent from the detailed description of preferred embodiments which follows, when considered together with the attached drawings and claims.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic representation of one implementation of the adipose tissue management system in accordance with the present invention.

FIG. 2 is a block diagram illustrative of fat cell subdivision utilization environment including a number of users, a set of medical practitioners, a fat cell subdivision management service, and a set of fat cell subdivision storage providers;

FIGS. 3A-3C are block diagrams of the fat cell subdivision utilization environment of FIG. 2 illustrating various interactions between users, medical practitioners, the fat cell subdivision management service, and fat cell subdivision storage providers.

FIGS. 4A and 4B are flow diagrams illustrative of the processes implemented by a medical practitioner in the generation of fat cell subdivisions for storage; and

FIG. 5 is a flow diagram illustrative of the processes implemented by a medical practitioner in the utilization of stored fat cell subdivisions in medical procedures.

FIG. 6 depicts a non-limiting embodiment of a fat cell injection device comprising a precision pump as described herein.

FIG. 7 depicts an additional non-limiting embodiment of a fat cell injection device comprising a precision pump as described herein

FIGS. 8A-8E depict non-limiting embodiments of a precision pump having adjustable volume as described herein.

FIGS. 9A-9E depict additional non-limiting embodiments of a precision pump having adjustable volume as described herein.

### DETAILED DESCRIPTION

Fat transfer (also known as autologous fat transplantation) involves transferring fat from one part of a person's body to another and typically involves some type of liposuction. Once the fat tissue has been acquired from one part of a patient's body, the fat is treated in a number of different ways (e.g. centrifugation, cleaned, drained). After treatment of the extracted fat tissue, the doctor can then either use the fat to immediately inject the fat cells back into the patient's body, or discard the fat cells as bio-waste. Areas of the body that are commonly injected with fat cells include; chest (particularly after radiation treatment), hands, penis, face, buttocks, and breasts. Therefore, the injection of fat cells can offer the benefit of enhancing a particular area of the body (e.g. breasts), or simply giving a particular area of the body a more youthful appearance (e.g. chest, hands, face) since age can atrophy fat cells giving a person a more skeletal appearance.

Aspects of the present disclosure include systems, methods and associated equipment for extracting fat cells from one part of a person's body, storing the fat cells by cryogenically freezing the fat cells (for short-term or long-term storage), and then utilizing the adipose stem cells for a future therapeutic application and/or thawing and injecting the fat cells back into the patient's body for cosmetic purposes.

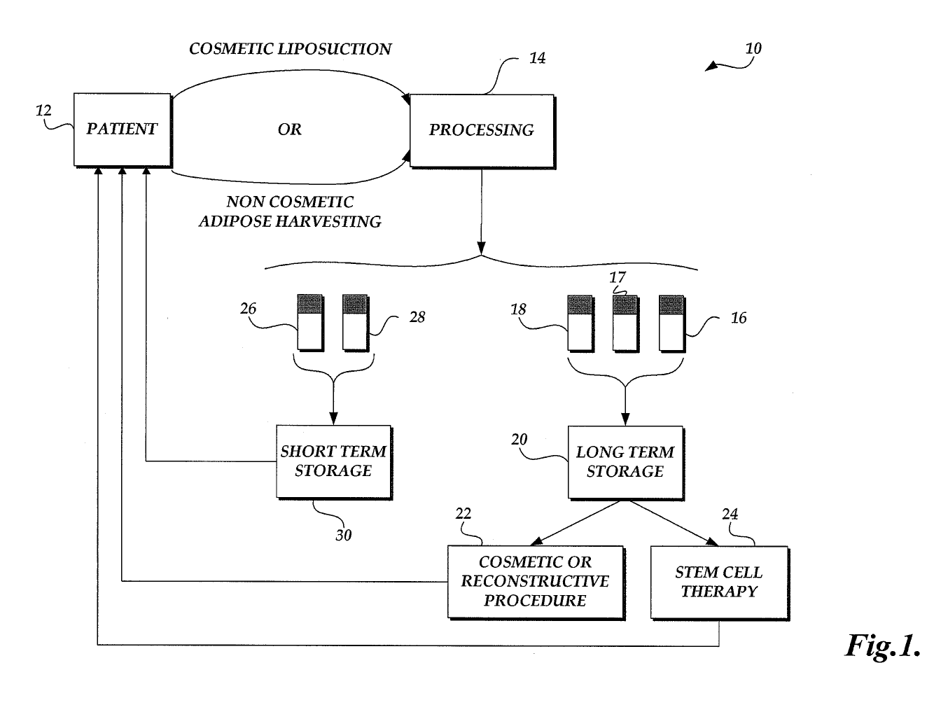

Referring to FIG. 1, there is illustrated a schematic representation of one adipose tissue management system in accordance with an aspect of the present invention. The system 10 includes a patient 12, from whom adipose tissue is harvested. Adipose tissue may be harvested as a byproduct of a cosmetic liposuction procedure, preformed under conditions adapted to promote survival of the harvested tissue. Alternatively, some patients may be interested in harvesting adipose tissue and/or stem cells for potential later use, but do not desire liposuction or other cosmetic remodeling at the present time. The volume of tissue needed to preserve stem cells is relatively small (e.g. one or two 100 ml bottles) compared to a typical liposuction, where the harvested tissue is measured more commonly in liters. Most patients have sufficient excess adipose tissue that a small volume can be harvested for future use without having a significant cosmetic impact.

The harvested adipose tissue is thereafter sent through processing 14, which includes a variety of potential process steps depending upon the intended storage and application. Processed adipose tissue will be stored in a plurality of vials 16, each vial containing what is sometimes referred to herein as a cell subdivision.

In accordance with the present invention, the adipose tissue may be directly advanced from the patient into the vials 16, in which case filtering, preservation or other processing technology is built into the vial. Alternatively, the adipose tissue is advanced through a processing stage and introduced into the vials thereafter.

A patient may elect to have tissue stored in a number of vials, such as at least about 2, 4, 10, 20 or more, depending upon the anticipated use later in time. The vials are preferably provided with engagement structures which allow the vial to be coupled directly to an effluent port on a processing apparatus, and also may subsequently be coupled directly to an influent port on an injection needle or injection gun for cosmetic reinjection into the patient. In this manner, the adipose tissue may be maintained in a closed system essentially from the point of extraction from the patient to the point of reintroduction.

The vials may all be treated in the same manner, or may be divided into two or more subgroups for different processing. In the illustrated system, a first set of vials 16, 17 and 18 are advanced into long term cryogenic storage 20. Vials may be maintained in long term storage 20 for any desired period, such as at least about 6 months or a year, up to 50 years or more prior to use. At a desired time, one or more of vials 16, 17 and 18 may be removed from long term storage 20 such as to accomplish a cosmetic procedure 22 on the patient 12. Cosmetic procedure 22, following a long term storage period of time may be in the nature of a lip augmentation, breast augmentation, augmentation of the back of the hands, of the face, or other procedure designed to achieve a cosmetic correction of the normal depletion of subcutaneous fat as a function of aging.

Following the long term storage period of time, one or more of the vials 16, 17 and 18 may be removed from storage and processed to accomplish a stem cell related therapy 24. As is understood in the art, a wide variety of therapeutic applications are under development, which depend upon or can be accomplished using adipose derived stem cells. Processes for extracting stem cells from adipose tissue and further processing the stem cells are known in the art and not reproduced herein. Typically, the stem cell therapy will be accomplished on the patient 12 from whom the stem cells were extracted (autologus), however that is not a requirement of the present system.

A second group of vials 26 and 28 may be prepared at the physician's office for short term local storage 30. Short term local storage 30 may include cryogenic preservation, or other, shorter term storage techniques known in the art. Vials 26 and 28 will be prepared for short term storage 30 in response to a request by the patient for a short term cosmetic procedure, such as a breast augmentation or other cosmetic injection that is to be accomplished shortly following the adipose tissue harvesting. Thus, a patient may return to the physician following liposuction for a series of visits, such as two or four or six or ten or more visits spaced apart such as by one or two or four or more weeks, for micro volume injections of harvested adipose tissue. The frequency and number of visits will be determined by the physician and the patient, depending upon the patient's healing response and the desired cosmetic result.

Further details of the foregoing system will be described below.

Subcutaneous fat may be removed from a patient using various liposuction techniques. The type of liposuction technique used may vary depending on the part of the body from which the fat is being extracted from and/or to acquire the extracted fat in a desired condition for its intended purposes. Therefore, any number of fat cell removal procedures (e.g. syringe withdrawal, fluid assisted or “wet” liposuction, tumescent liposuction, non-fluid assisted or “dry” liposuction, ultrasound assisted liposuction) may be used to extract fat cells from one part of a person's body for it to be transferred to another part of the same person's body without departing from the scope of the present invention. Although a wide variety of liposuction techniques exist in the prior art, a number of these techniques are destructive to adipose tissue and are instead optimized for bulk tissue removal. Thus, certain tissue harvesting techniques may be preferred where, as here, survival of the harvested tissue is desirable. Certain currently available equipment such as an ultrasound based Vaser® Liposelection System by Sound Surgical Technologies LLC, or water jet based Body-Jet® by Ellipsemed Limited, may be suitable for use in the context of the present systems.

Additionally, a low pressure suction device can be used for extracting fat cells and maintaining their cell structure and viability. Furthermore, typically no smaller than a 14 gauge blunt tipped needle can be used for extracting the fat tissue in order to prevent clogging and damaging the fat cells. The blunt tip on the needle is to avoid piercing any arteries or veins.

The tissue transfer process for either large or small volume harvesting includes selecting an area as the donor site (e.g., the medial area of the knee, the abdominal area, or the trochanteric area) and then infiltrating the area with a cold saline solution with the addition of about 15 cc of adrenalin and about 20 to about 30 cc of lidocaine 0.5% per 500 cc. Adipose tissue may be removed in small volumes using a cannula with a 2 mm diameter and a 3 cc syringe. The syringes may be placed directly in a centrifuge set at about 2700 rpm and run for 15 minutes, resulting in separation of the purified adipose tissue for injection from its water content and from oil resulting from the destruction of damaged adipocytes. The oil and residual liquid (including triglycerides) may be discarded.

Fat cells removed from a part of a person's body are preferably transferred directly to a sterile container that can at least store the fat cells in a sterile environment and does not allow any external exposure to the contained fat cells. One or two or three or four or five or ten or more sterile containers may be utilized, to store the fat cells removed from a single liposuction procedure. The containers may have a volume of no more than about 50 cc, 100 cc, 150 cc, or 200 cc, depending upon the anticipated future use. Dividing the sample tissue into a plurality of sealed containers allows the containers to be reutilized at different points in time, without breaking the sterile seal on the unused containers. The containers may be preloaded with a volume of one or more additives, such as will be described in further detail below. The sterile container may include features that allow fluids surrounding the fat cells to be filtered away and possibly discarded out from the storage container by sterile means so as to not contaminate the fat cells within the sterile container. Processes such as centrifugation may be performed on the sterile container to increase the separation of fluids and solids so that, for example, the fat cells can be further filtered and unwanted parts can be removed.

In accordance with other aspects of the present disclosure, the fat cell subdivisions may be processed after extraction. For example, oxygen may be provided to the fat cells before or while contained in the sterile container. For example, it has been contemplated that the fat cell suction device (used to perform the liposuction) could inject a supersaturated oxygen solution into the fat cells before extraction in order for the supersaturated oxygen solution to diffuse into the fat cells and aid in the preservation of their viability. The ability of the fat cells to maintain their viability is in part due to their ability to stimulate angiogenesis, which allows blood supply to nourish surrounding fat cells. Well oxygenated fat cells are able to undergo angiogenesis with greater success than fat cells which have experienced a significant deprivation of oxygen. Therefore, it is an advantage of the present method to incorporate means for minimizing the oxygen deprivation of the extracted fat cells.

In addition to harvesting the adipose tissue, the procedure for procurement and treatment of autologous (or other donor) adipose tissue or lipoaspirate may include purifying the tissue. The lipoaspirate purification procedure is generally designed to remove a large part of the triglyceride stored in the harvested adipose tissue. The purification by centrifugation or similar techniques also functions to cause lesions in the thin cytoplasmic sheets of mature adipocytes in the harvested adipose tissue. In other words, the purification may include intentionally causing additional damage to the adipocytes that have been traumatized by liposuction or harvesting processes, and this additional damage is preferably to the point of one or more lesions so as to enhance the speed at which a treated patient is able to clear the damaged mature adipocytes after implant.

In some embodiments, purification is obtained by centrifugation carried out, in part, to separate a set of adipose tissue (i.e., the purified adipose tissue) from its water content and from the oil produced by the destruction of the damaged adipocytes. An advantage of this purification technique may be that there is no need for the use of additional cell culture steps to grow additional tissue outside the patient's body as was common with many other tissue implant techniques, thus avoiding culturing, better controlling risks of micro-organism contamination, reducing the complexity of the tissue preparation process, and controlling or limiting associated costs. A further advantage of the purification or tissue preparation process is that by the process does not require the technically challenging step of isolating or extracting adipose-derived stem cells (ADAS) but instead allows the ADAS to remain in their natural support structure or 3D scaffold which facilitates vascularization and other benefits. Additional detail can be seen in US patent application publication No. 2009/0181104 to Rigotti et al., the disclosure of which is incorporated in its entirety herein by reference.

There are a number of additional processing procedures that may be conducted to best condition the fat cells for at least one of short-term storage, long-term storage, and injection of fat cells back into the patient's body. For example, preservatives may be added to the fat cells in order to provide an enhancement (e.g. increased viability) either while the fat cells are being stored and/or once the fat cells are injected back into the patient's body. Any number of preservatives and/or additives currently in use can be deposited into the sterile container to interact with the contained fat cells without departing from the scope of the present invention.

Further examples include the addition of stem cells (which have been previously separated by known methods from other extracted fat cells) to the fat cells contained within the sterile container. It is also possible to add the additives (e.g. preservatives, stem cells) at any point necessary (e.g. before or after storage) for producing the desired outcome of the treated fat cells contained within the sterile container. The sterile container includes features such as a sterile inlet port for enabling the additives to be added to the fat cells contained within the sterile container. The sterile container may also include features that allow solvents to be added and dispensed in order to wash or rinse the fat cells, if desired. The features of the sterile container that enable the addition and removal of solvents are such that sterile conditions are maintained within the sterile container at all times. By way of example only, luer-locking ports would enable an enclosed fluidic pathway for solvents to be added to the sterile container, thus minimizing contamination within the sterile container.

Any of a wide variety of additives may be added to the adipose cells, depending upon the intended storage protocol and use. For example, biologically active agents that may be added to the cells to be transplanted include, but are not limited to, antioxidants, vitamins, membrane stabilizers, minerals, osmotic protectants, coenzymes, viscosity enhancers, hormones, and growth factors. Numerous mechanisms have been implicated in the cause of cell death in transplanted cells, for example, membrane disruption and free radical formation. Antioxidants may be used in fat transplantation to reduce free radical formation. Antioxidants scavenge free radicals and prevent damage caused by reactive oxygen species. The antioxidants may be enzymatic or nonenzymatic antioxidants. Enzymatic antioxidants include, for example, superoxide dismutase, glutathione peroxidase, and catalase. Exemplary non-enzymatic antioxidants include alpha-tocopherol (vitamin E), vitamin A, glutathione, carotenoids (e.g., lycopene, lutein, polyphenols, .beta.-carotene), flavonoids, flavones, flavonols, glutathione, N-acetyl cysteine, cysteine, lipoic acid, ubiquinal (coenzyme Q), ubiquinone (coenzyme Q10), melatonin, lycopene, butylated hydroxyanisole, butylated hydroxytoluene (BHT), benzoates, methyl paraben, propyl paraben, proanthocyanidins, mannitol, and ethylenediamine tetraacetic acid (EDTA). In certain embodiments, the antioxidant is a metallic antioxidant. In certain embodiments, the antioxidant is selenium. In certain embodiments, the antioxidant is zinc. In certain embodiments, the antioxidant is copper. In certain embodiments, the antioxidant is germanium.

The adipose cell additive may further comprise a vitamin. The vitamin may be an antioxidant. In certain embodiments, the vitamin is alpha-tocopherol (vitamin E). In certain embodiments, the vitamin is coenzyme Q10. In certain embodiments, the vitamin is beta-carotene. Other vitamins that may be added include one or a combination of two or more of vitamin A, vitamin B.sub.1 (thiamine), vitamin B.sub.2 (riboflavin), vitamin B.sub.3 (niacin), vitamin B.sub.4 (adenine), vitamin B.sub.5 (pantothenic acid), vitamin B.sub.6 (pyridoxine), vitamin B.sub.7 (biotin), vitamin B.sub.9 (folic acid), vitamin B.sub.12 (cyanocobalamin), vitamin D (ergocalciferol), and vitamin K.

The adipose cell additive may further comprise a hormone or growth factor. In certain embodiments, the hormone or growth factor is insulin, glitazones, cholesterol, VEGF, FGF, EGF, PDGF, etc. In certain embodiments, the additive further comprises an organic acid (e.g., lipoic acid). In certain embodiments, the additive may comprise a thiol-containing or disulfide-containing molecule (e.g., lipoic acid, glutathione), and/or a small organic molecule (e.g., anthocyanins, capsaicins).

Formulations including adipose cells and additives may comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular formulation desired. Remington's The Science and Practice of Pharmacy, 21.sup.st Edition, A. R. Gennaro, (Lippincott, Williams & Wilkins, Baltimore, Md., 2006; incorporated herein by reference) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of the adipose cell and additive compositions include, but are not limited to, inert diluents, dispersing agents, surface active agents and/or emulsifiers, disintegrating agents, preservatives, buffering agents, lubricating agents, and/or oils. Excipients such as coloring agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus®, Phenonip®, methylparaben, Germall 115, Germaben II, Neolone™, Kathon™, and Euxyl®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Once the fat cells contained within the sterile container subdivisions have been treated (e.g. centrifugation, filtration, addition of preservatives, other additives and/or stem cells, washed/rinsed), the fat cell subdivisions may then be at least one of long-term stored, short-term stored, injected back into the patient at a selected part of the patient's body, or discarded as bio-waste. The sterile container is configured such that it is suitable for carrying out any of the aforementioned steps. For example, the sterile container is composed of materials that are suitable for long-term storage which may require exposure to various gasses, liquids, pressures and temperatures. Additionally, the sterile container is easily adaptable to a device which can deliver the fat cells contained within the sterile container directly (with the exception of any necessary dispensing needle and/or tubing) into the patient. This minimizes the fat cell's exposure to potentially non-sterile conditions and maintains optimal sterile conditions for the transport of the fat cells from the sterile container into the patient's body.

The present method of fat transfer includes the option of storing the fat cells extracted from a patient for a long period of time than what has been previously contemplated or attempted by those skilled in the art, and which will be disclosed in detail herein. Once the fat cells have been properly contained and prepared (e.g. rinsed, additives added, etc.) within the sterile container, the sterile container is then properly labeled. The label should allow the sterile container to be stored for a long period of time and enable the patient or doctor to retrieve the fat when necessary or desired. For example, the sterile container can be labeled and the corresponding information entered into a tracking system such that several or many years may pass by and the patient is able to retrieve his or her own fat cells, and not be accidently given fat cells from another patient.

A number of complications can occur if a patient is injected with fat cells from another patient, which could result from poor labeling and tracking of the sterile containers. Illustratively, an electronic tracking system such as an RFID, bar code or other system may be used to associate the sterile containers with the correct patients. RFID is well known as a secure method of tracking and labeling which can easily be implemented to a computer tracking system that can communicate to a vast number of computer stations around the world via the internet. Additionally, it is contemplated that a locking feature can be integrated into the sterile container such that only the physician who extracted the cells can access the sterile container for injecting the fat cells back into the patient. If there is any desire or need to change physicians, it would require a strict and confidential exchange of patient records (e.g. RFID's, ID codes) to the new physician in order for the new physician to be able to obtain and use the sterile container. Again, this would be a method to further ensure that the correct fat cells are being injected into patients. Any number of labeling, tracking, and locking methods and mechanisms can be used without departing from the scope of the present invention as long as correct locating and identification of sterile containers are successfully accomplished so that the contained fat cells are delivered into the correct patient. One suitable storage and retrieval system is disclosed in U.S. Pat. No. 6,564,120 to Richard, et al., the disclosure of which is incorporated in its entirety herein by reference.

After proper labeling of the sterile container, the sterile container (with the contained extracted fat cells) are either shipped to a remote long-term storage facility within 12 hours so the sterile container can be placed into long-term storage (cryopreserved), or the sterile container is cryogenically frozen at the fat extraction facility. If a sterile container is cryogenically frozen at the fat extraction facility (e.g. doctor's office) it can then either be stored at the fat extraction facility in their cryogenically frozen state, or transported in their cryogenically frozen state to a remote long-term storage facility. It is generally necessary that the sterile container is cryopreserved (as will be discussed in detail below) within 12 hours of having been extracted from the patient and that the sterile container is maintained in a cryogenically frozen state until the fat cells are to be used (e.g. stem cell extraction, fat grafting). During the time between fat extraction and preparing the sterile container containing the extracted fat cells for cryopreservation (which should be no more than 12 hours), it is recommended that the sterile container be kept in a cool environment (e.g. maintained at 4 deg C. in an igloo).

Preparation of the fat cells for long-term storage depends on the equipment available at the facility where the fat cells were extracted and the type of transportation system that will be used to transport the fat cells from the place of extraction to the remote long-term storage facility. It is also contemplated that the sterile containers containing a person's extracted fat cells could be stored at more than one long-term storage facility (e.g. 2 different long-term storage facilities each storing 2 sets of sterile containers) in order to retain at least a portion of the extracted fat cells if a catastrophic event were to occur at one of the long-term storage facilities.

Long-term storage of the fat cells contained within the sterile containers includes the step of cryogenically freezing (or cryopreserving) the fat cells. Whereas simply freezing fat cells disrupt the fat cells and result in poor viability, cryogenically freezing the fat cells result in optimum viability (over 95% viability of fat cells can be achieved). Cryogenically freezing the fat cells involves slowly bringing the temperature of the fat cells down to approximately −196 deg C. It is also recognized that a temperature other than approximately −196 deg C. may be more suitable for cryogenically freezing fat cells once further research has been conducted, but a change in temperature will not depart from the scope of this invention disclosure. Current cryogenic methods include using liquid nitrogen to assist in lowering substances (e.g. blood samples, sperm) to temperatures of −196 deg C. and maintaining these substances at −196 deg C. in liquid nitrogen freezers. Bringing the fat cells down to a temperature of −196 deg C. requires a slow rate of cooling in order to not damage the fat cells and maintain their viability. It is known to those skilled in the art of cryogenically freezing mammalian cells that a typical cooling rate of approximately 1 degree C. per minute is appropriate, but factors such as cells differing in size and water permeability can affect the cooling rate. Therefore, the cooling rate of the fat cells may require altering the rate of cooling (and subsequent thawing) without departing from the scope of the present invention.

Machines (e.g. controlled-rate cooling machines) to assist in the cooling of the fat cells are preferably used to ensure proper cooling rates since fat cells that are cooled at improper cooling rates can significantly affect the viability of the fat cells. The sterile containers can be cryogenically frozen using a number of appropriate cooling machines either before (e.g. at the doctor's office) or once at the long term-storage facility. Additionally, cryoprotectants (e.g. glycerol, dimethyl sulfoxide) and vitrification techniques are utilized, as necessary, that aid in the preservation of the fat cells and promote their viability.

Once the fat cells contained within the sterile containers have been brought to the appropriate temperature for cryogenically freezing the fat cells, the sterile containers can then be placed into a long-term storage compartment (e.g. liquid nitrogen freezer) which holds the temperature of the fat cells at approximately −196 degrees C. The fat cells are kept at this temperature for as long of a time as necessary (e.g. several decades) while maintaining their ability to be implanted back into the patient when desired.

Cryogenically freezing is currently utilized to preserve such things as blood and sperm, but the process of cryogenically freezing fat cells for the use of later implanting the fat cells back into the patient from which the fat cells were extracted from is not presently available. An advantage of the techniques of the present disclosure allows a person to extract healthy fat cells from their body and have the fat cells stored until the person desires or requires the injection of the fat cells back into their body. For example, a person could remove excess fat from one part of their body (e.g. abdomen, thighs) and store the fat so that it can be used several years or decades later to reconstruct a breast that underwent a lumpectomy. The cryogenically stored fat cells can be injected into the area where the breast tissue was removed from the lumpectomy, resulting in an improved appearance in the breast without the use of synthetic implants.

Further benefits include the naturally occurring stem cells which are prominent in fat cells. Potential rejuvenation of surrounding cells and improved viability of the injected fat cells can result from the presence of stem cells. Furthermore, the younger the fat cells are extracted, the younger the stem cells within the fat cells are, thus the stem cells will have a greater potential for providing the above mentioned benefits. Therefore, by extracting fat cells at a younger age (e.g. 20's and 30's) and having the ability to cryogenically freeze the fat cells for future use, the present invention will allow several benefits to people which are currently unavailable.

Another example is if someone were to undergo radiation in their later years (40's 50's) due to cancer. Radiation treatment can atrophy fat cells and give a sunken appearance to a person's features, particularly the face and chest area. If the person had cryogenically frozen fat cells from their body in their 20's or 30's, the person could have these fat cells injected (once thawed and prepared) into their face and chest to regain their more healthy and youthful appearance. Similarly, a person could use the cryogenically stored fat cells to inject their fat cells back into their face and hands, for example, for cosmetic reasons since age tends to atrophy fat cells in these areas and give a person an older appearance. Therefore, fat cell injection can be used for both reconstructive and cosmetic purposes.

In order for the cryogenically stored fat cells to be used for injection back into the person from which the fat cells were extracted from, the correct sterile container containing the fat cells must be located. Once properly located, the fat cells are transported to the fat injection facility where the patient will be injected with the fat cells. The fat cells must either transported in their cryogenic state (at −196 deg C.) or in a cooled environment (e.g. at 4 deg C.) for no longer than 12 hours. How the fat cells are transported is partially dependent upon the duration of transportation of the fat cells since the fat cells must not be thawed from their cryogenically frozen state for no longer than 12 hours. Additionally, the equipment available during transportation (for maintaining necessary temperatures), and the equipment available at the fat injection facility (for thawing the fat cells) also depend on how the fat cells will be shipped.

Before the fat cells contained in the sterile containers can be used, the sterile container must be placed into a machine which increases the temperature at a slow and controlled rate, similarly as to what was described above for cryogenically freezing the fat cells. Preferably, facilities would be equipped with a cooling machine which would both bring the sterile container down to cryogenically frozen temperatures (as described above), which should also be able to bring the cryogenically frozen sterile container back to a desired temperature. The desired temperature would be dependent upon whether the sterile container will need to be stored at a cool temperature for use within 12 hours, thus only bringing the sterile container up to a temperature of approximately 4 deg C., or if the fat cells are to be used immediately. If they are to be used immediately, the sterile container can be brought up to average room temperature of approximately 20-25 deg C.

As mentioned above, the sterile container is configured such that it can be coupled directly to a fat injection device which promotes optimum sterility of the fat cells by not requiring unnecessary transferring of the fat cells and eliminating exposure of the fat cells to the environment. Furthermore, it is preferred that the sterile containers contain no more than about 200 cc's of fat cells, in some embodiments no more than about 100 cc's since no more than about 200 cc's would typically be injected into a body part in a single serial fat injection session (in order to allow angiogenesis to occur). In the circumstance where a patient has more than 200 cc's of fat cells removed, it is recommended that multiple sterile containers are used. Ultimately, this reduces the potential for fat cells to be wasted since once the cells have been removed from their cryogenically frozen state they can either be used (e.g. injected into the body, stem cells extracted) or discarded as bio-waste since it is not recommended that the fat cells be re-cryogenically frozen.

Therefore, if a person wanted a simple facial injection (which typically only requires approximately 25 cc's of fat cells), there would typically only be a need to transport one sterile container containing approximately no more than 200 cc's of fat cells instead of, for example, the entire two liters of fat cells that were extracted from the patient during a single liposuction procedure. If a patient believed it likely that small volume procedures may be desirable down the road, they may want to store harvested adipose tissue in a variety of different volumes. For example, a first group of vials having a first volume such as two or four or six or more vials having no more than about 25 cc's or no more than about 50 cc's may be utilized. From the same liposuction procedure, at least a second group of vials having a second volume such as at least about 100 cc's or at least about 200 cc's may also be processed and stored. Preserving the tissue in at least two or three or more different volume containers may increase processing costs, but may minimize the risk of waste that might result from a future procedure. However, any size and shape sterile container can be used without departing from the scope of the present invention.

It is also contemplated that the sterile container would have either features or combined devices that would allow the outer surfaces of the sterile container to remain sterile. Since the sterile container is placed in non-sterile environments during storage and is unable to be sterilized (due to the contained fat cells), the sterile container would not be able to be re-introduced into a sterile surgical room for the implanting of the fat cells within the sterile container. By way of example, the sterile container could mate with a complementary sealed enclosure such as a sterile bag. The sterile bag could accommodate any number of the features of the sterile container for allowing the sterile container to undergo at least all of the processes and methods disclosed herein. The sterile bag could protect the outer surfaces of the sterile container from being contaminated, and then could be removed just prior to introducing the sterile container into a sterile field. Another example would be to have a double-walled sterile container where the outer contaminated container wall could be removed just prior to introducing the sterile container into a sterile field. Once the sterile container (with a sterile outer wall) is introduced into the sterile field, it can then be inserted into or otherwise coupled directly to a sterile fat injection device. Alternatively, the sterile container (with non-sterile outer walls) could be placed in communication with a pump from somewhere outside of the sterile field which would be connected to a fat injection device that is in the sterile field.

Once the sterile container is correctly adapted to a fat injection device, the fat cells from within the sterile container are injected into the patient's body in multiple small increments. It is currently known in the art that injecting fat cells in approximately 0.25 mL to 0.5 mL volumes into the body allows the injected fat cells to undergo angiogenesis and maintain their viability. Therefore, multiple fat injections are required in a single fat injection session. More than one fat injection session of multiple fat injections may also be required in order to achieve the total desired injection of fat cells. It is recommended that no larger than a 2 mm blunt tipped needle is used.

Another benefit of the current invention includes requiring minimal liposuction procedures for performing multiple fat injection sessions over potentially a person's lifetime. Although liposuction is a relatively safe procedure, it comes with its associated complications and risks which make it beneficial for a person to have as few liposuctions as possible. Therefore, if a person only requires a single liposuction procedure for more than one fat injection session performed within the person's lifetime, then a significant amount of physical complications and risks would be avoided. Additionally, the discomfort and recovery time associated with undergoing a liposuction procedure would be minimized with the present invention.

In one embodiment, cosmetic soft tissue augmentation such as of the face, neck, hands, breasts or elsewhere on the body can be performed using Bircoll's Fat Transfer techniques either at the time of harvesting (within days, weeks or months) or years or decades later. Bircoll's Fat Transfer techniques include administering multiple microinjections of fat cells in desired areas of reconstruction and/or enhancement. The volume of fat injected is determined by the blood supply of the recipient area, i.e., the defect size and its effective healed state. In a properly healed state with the required lapse time of approximately 3-4 months, revascularization should have occurred, thus making the site receptive to Bircoll's Fat Transfer procedure. The injection of fat is performed within Bircoll's Fat Transfer parameters. One skilled in the relevant will understand the various parameters and procedures associated in the illustrative Bircoll's Fat Transfer techniques, which are incorporated herein.

Illustratively, Bircoll's Fat Transfer procedure involves injecting liposuction removed fat cells in multiple small volumes (e.g., 0.25 mL-0.5 mL) over potentially multiple sessions. During each fat injection session, the total volume of fat injected into a particular area varies. By way of example, approximately 200 mL of total fat cells may be injected during a single session for a breast reconstruction/augmentation. Multiple sessions may be required in order to achieve larger total volumes of fat injected. The advantage of Bircoll's Fat Transfer procedure is that the multiple small injections of fat (e.g., 0.25 mL-0.5 mL) are able to undergo angiogenesis, thus allowing the fat cells to remain viable and not calcify. However, in some embodiments, larger or smaller volumes may be injected. For example, in some embodiments, injection volume (for each individual injection) can range from about 0.1 mL to about 0.2 mL, from about 0.2 mL to about 0.3 mL, about 0.3 mL to about 0.4 mL, from about 0.4 mL to about 0.5 mL, from about 0.5 mL to about 0.6 mL, from about 0.6 mL to about 0.7 mL, from about 0.7 mL to about 0.8 mL, from about 0.8 mL to about 0.9 mL, from about 0.9 mL to about 1.0 mL, and overlapping ranges thereof. Proper instrumentation for microinjection of fat cells is necessary, as described in Bircoll's Fat Transfer, and is also incorporated herein. In several embodiments, unit volumes of adipose tissue are delivered. In some embodiments, a plurality of unit volumes are delivered in a single session. Generally, the unit volumes are no greater than about 1.5 mL, and in some implementations, no more than about 1.0 mL, 0.75 mL, 0.5 mL or 0.25 mL, depending on the desired clinical result. In several embodiments, the instrumentation is designed to insure maximum viability of the grafted fat tissue. As such, in some embodiments, a specific volume (e.g., a micro-volume) is injected in order to maximize the viability of the delivered fat cells. The injection of multiple small volumes at various sites is advantageous because it allows for the deposition of a volume (and therefore a number) of cells that can be supported by the existing and newly forming tissue infrastructure (e.g., blood vessels etc.) and therefore remain viable. Moreover, the deposition of multiple small volumes of fat cells allows for a fine tuning of the structure and shape the breast tissue.

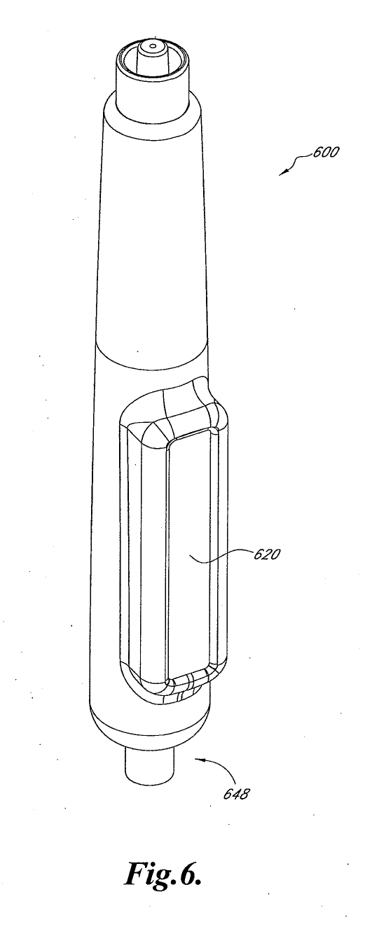

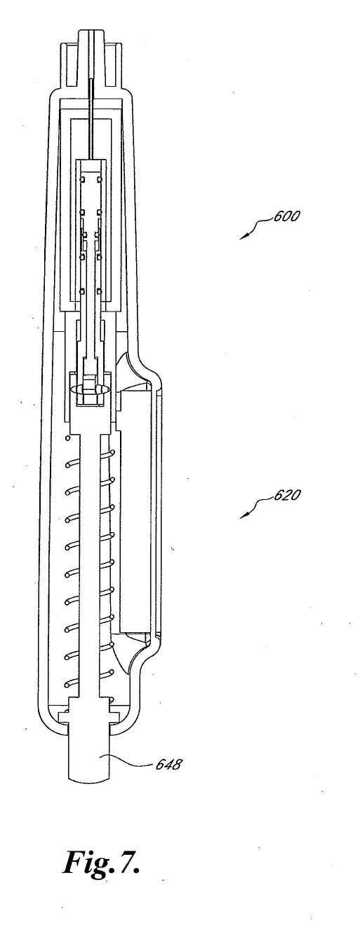

In several embodiments an injection device comprising one or more chambers and one or more precisions pumps (to expel the fat cells) is employed to inject fat cells into recipient breast tissue. For example, an injection device can comprise, in some embodiments a pistol grip style injection gun that is operated by a medical provider via a trigger or other equivalent mechanism. In some embodiments, a wand or pen is employed, such devices having an actuator to dispense fat cells from chambers within the device (non-limiting embodiments are depicted in FIGS. 6 and 7).

In some embodiments, the injection devices comprise precision pumps that are advantageously reversible, self-priming, positive displacement, disposable, and capable of pumping viscous fluids and, as such, are suitable for applications such as fat transfer including collection, delivery and re-depositing through appropriately sized lumen. In some embodiments, the precision pumps are operated manually (e.g., the dispensed volume is determined by medical personnel, while in other embodiments, the pumps and injection devices are programmably operated.

FIGS. 6 and 7 illustrate a non-limiting example of a precision pump 600, which is incorporated into a hand-held injection device comprising a manual actuator 648. While not shown in FIGS. 6 and 7, in some embodiments, a device having a rounded, ovalized, or otherwise blunt delivery tip is used to deliver fat cells, in order to minimize tissue trauma during the procedure (e.g., the instruments optionally function as blunt dissectors). The pump 600 may be provided with a distal luer (or other similar) connector for receiving a complementary connector on a standard small gauge hypodermic needle. In some embodiments, multiple outlets (e.g., at least 2, at least 4, at least 6, or more) are present in a distal portion of the injection device in order to accomplish a plurality of fat cell depositions within a target delivery space. Advantageously, the presence of multiple outlet ports provides a redundancy that maintains the ability of the device to deliver fat cells, even if an outlet becomes clogged with tissue. In some embodiments, the multiple outlets are chamfered to minimize damage to tissue during the injection procedure. However, in some embodiments, single output outlets are present in a device.

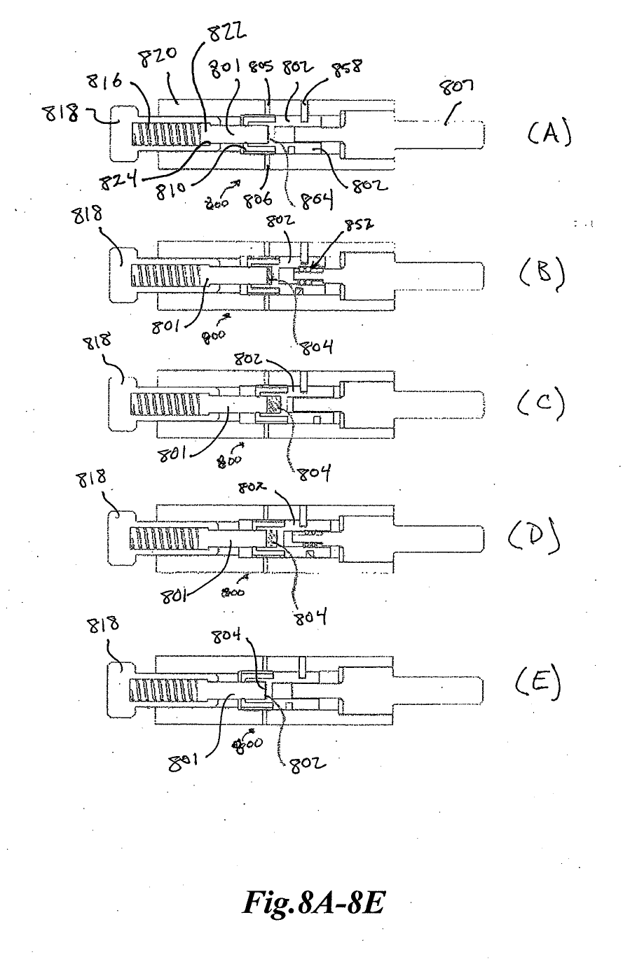

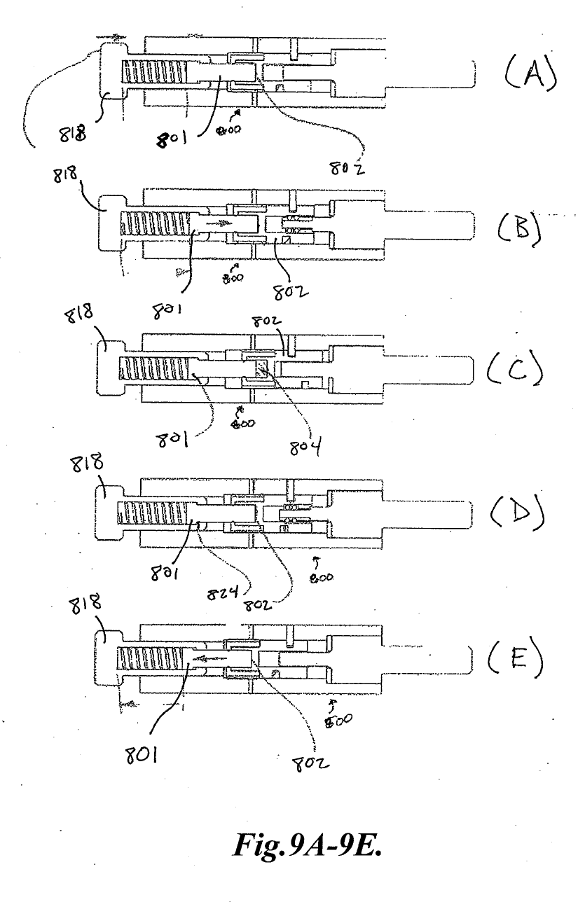

In some embodiments, the injection devices comprise a single chamber (e.g., a reservoir) for holding a volume of fat cells to be injected into a recipient. Such devices further comprise a precision pump that allows for the repeated injection of micro-volumes (e.g., volumes ranging from about 0.25 mL to 0.5 mL per injection site) until the reservoir volume has been depleted. Some pumps employed in injection devices comprise an adjustable delivery volume, examples of which are illustrated in FIGS. 8-9. However, in some embodiments, single chamber single injection devices are used. Likewise in some embodiments, multiple chamber multiple injection devices are used (e.g., each chamber is used once in a serial series of fat cell injections).

FIGS. 8A-E and 9A-E illustrate non-limiting examples of precision pumps 800 that include displacement cavities having adjustable volumes. As illustrated, a cavity adjustment feature enables the adjustment of the maximum volume of the cavity formed within the pump. The pump 800 comprises a spring loaded piston post or pin 801 that dictates the volume of the cavity formed within the precision pump 800 and can be adjusted to reduce or enlarge the cavity 804 formed at the furthest (retracted) extent of the stroke piston 802. Therefore, the volume of fluid (e.g., fat cells) that is transferred through the precision pump 800 with each stroke of the piston can be tuned or calibrated to the desired dispensing volume (for example, about 0.25 to about 0.5 mL per injection site).

The precision pump 800 comprises a spring-loaded pin or plunger 801. The pin or plunger 801 is movably positioned within the housing 820. A cap, plug, or stop 818 is adjustably attached to the housing 820. A spring 816 is positioned within the housing 820 to engage the stop 818 and the pin 801 on a side of the pin that is opposite the piston 802, and urges the pin 801 toward the piston 802.

The pin 801 and stop 818 can be configured such that when the pin 801 is fully advanced the pin is seated against stop 818. For example, the pin 801 can comprise a shoulder 822 and the stop 818 can comprise a rim 824, the shoulder and the rim being sized and shaped to engage one another when the pin 801 is advanced by the spring 816 toward the rim 824. Thus, movement the stop 818 into or out of the housing 820 adjusts the maximum distance the pin 801 can advance in the direction of the piston 802.

FIGS. 8(A) and 9(A) show the piston 802 in a position of farthest advancement toward the stop 818. FIGS. 8(B) and 9(B) show the piston 802 in a position retracted from the position of farthest advancement toward the stop 818. FIGS. 8(C) and 9(C) show the piston 802 in a position farthest retraction from the pin 801. FIGS. 8(D) and 9(D) show the piston 802 in a position advanced toward the pin 801 from the position of farthest retraction from the pin 801. FIGS. 8(E) and 9(E) illustrate the piston 802 returned to the position of farthest advancement toward the stop 818.

FIGS. 30(A)-(E) illustrate operation of the pump 800 when the stop 818 is adjusted for transfer of a maximum volume of fluid. As illustrated in FIG. 30(E), when the piston 802 is at its most advanced position in the direction of the stop 818, the cavity 804 has closed and has no volume, or approximately no volume.

FIGS. 9(A)-(E) illustrate operation of the pump 800 when the stop 818 is adjusted for transfer of less than a maximum volume of fluid. As illustrated in FIG. 9(D), before the piston 802 is at its most advanced position in the direction of the stop 818, the cavity 804 has closed and has no volume, or approximately no volume. If the spring-loaded pin or plunger 801 engages the piston 802 and the piston 802 is advanced toward the stop 818, the plunger 801 will move with the piston 802 against the force of the spring 816 to retract into a recess in the stop, as illustrated in FIG. 9(E), for example.

The stop 818 can comprise threads that cooperate with threads of the housing 820 for adjustment of the stop 818 relative to the housing 820. Other types of connections between the stop 818 and the housing 820 can be used in some embodiments.

The stop 818 can be adjusted during manufacturing for precision of fluid transferred with each revolution then fixed or be adjustable by a user to vary the rate of flow of the pump. In some embodiments, a fluid flow meter can be connected to an outlet of the pump and the stop 818 can be adjusted until the desired flow rate is attached. In some embodiments, the maximum volume of the cavity can be adjusted during operation of the pump. In some embodiments, the pump can comprise indicators corresponding to specific fluid flow rates to facilitate adjustment after manufacturing.

Additional information related to such precision pumps for fluid (e.g., fat cell) delivery, and their incorporation into delivery devices can be found in PCT Application No. PCT/US2010/051707, filed Oct. 6, 2010 and U.S. Provisional Application No. 61/249,145, filed Oct. 6, 2009, the disclosure of each of which is incorporated by reference herein. It shall be appreciated that in some embodiments, a precision pump need not be incorporated into a delivery device (e.g., the pump can optionally be a separate device). In such embodiments, the connections between a pump delivering fat cells to target tissue via the injector device can comprise any variety of secure and fluid tight fittings known in the art (e.g, threaded fittings, luer lock fittings, press-fit couplers with o-rings, and the like).

The stored fat cells can alternatively be utilized for soft tissue reconstruction following a tissue excision such as for diagnostic (biopsy) or therapeutic (cancer excision) purposes. For example, fat cells can be serially injected in stages (i.e. Bircoll's Fat Transfer technique) into the breast to gradually fill in the defect left by the preventive breast cancer excision or to perform a purely cosmetic soft tissue enhancement. The defect left by the excision of the breast tissue cannot be replaced immediately with fat cells due to swelling, bleeding and cavitations of the surrounding excised area. In some embodiments, an initial injection of fat cells (e.g., Bircoll Fat Transfer) for reconstruction of the breast can occur within several weeks (e.g., 1-2 weeks, 2-3 weeks, or 3-4 weeks). Subsequent injections could be performed after a period of several months (e.g., 1-2 months, 2-3 months, 3-4 months, or longer). In some embodiments, this delay is advantageous in that it allows sufficient time for angiogenesis to vascularize the tissue, thereby supporting the viability of the injected cells and tissue damaged by the surgery. Some surgeries require the implantation of drains to allow for the release of accumulated fluid post-surgery. In such embodiments, additional injections of fat cells could be performed in about 3 months post-removal of the drains (e.g., 1-2 months, 2-3 months, 3-4 months, or longer). Due to this period of time to allow for revascularization to occur before the Bircoll Fat Transfer is performed, proper handling of the fat cells before, during and after storage is necessary to maintain the optimal viability of the fat cells.

In one embodiment, if excess fat cells are removed than required to repair the defect left by the breast tissue excision, the fat remaining fat cells may be used at any time in other areas of the patient's body. Fat injection is well known in the art for use in cosmetic surgery. By way of example, the excess stored fat may be used to enhance the patient's facial features at a later time. Therefore, the excess fat cells may be at least used for cosmetic purposes, reconstructive uses, or future stem cell extraction, thus providing even further benefits to the patient. In other embodiments, fat cell extraction may be performed for purposes of preparing fat cell subdivisions for later use.

The present invention also contemplates prophylactic treatment such as the prevention or reduction in the risk of breast cancer. At least about 95% or more of all breast cancer originates in the epithelial cells that line the interior of the intraductal system. Progression occurs through a well defined series of stages, taking, on average, eight years before the cancer can be detected by mammography or up to ten years before the lesion is manually palpable. The epithelial lining progresses from an initial hyperplasia to an atypical ductal hyperplasia which is considered a “precancer” stage. Atypical hyperplasia may progress to ductal carcinoma in situ, and onto invasive ductal carcinoma.

At the present time, patients who know that they are in a high risk group are largely limited to watchful waiting, as few options are available for high risk patients to reduce their risks. Patients who have already had breast cancer, or a combination of other risk factors and who are beyond child bearing age essentially have prophylactic mastectomy as the only available risk reduction procedure.

Thus, in accordance with the present invention, a less invasive method is provided of reducing or eliminating the risk of breast cancer while preserving cosmetic appearance of the breast. The method includes the steps of removing the intraductal system, included within functional tissue (also referred to as the parenchyma), leaving the remainder of normal tissue intact. The cavity caused by the parenchyma incision is thereafter filled or adjacent tissue is filled using the fat transfer techniques described elsewhere herein. Parenchyma excision may be accomplished in any of a variety of ways, such as via an inferior fold incision, a proximal incision, or other access methods established in the art. For example, in some embodiments, a balloon dissector can inserted via a proximal incision and be used to create tissue planes in order to facilitate the separation of ductal tissue to be excised from the ductal tissue that would be spared. Access tools for achieving a trans-umbilical access that may be utilized to remove parenchyma tissue is disclosed, for example, in U.S. Provisional Patent Application No. 61/331,711 to Bircoll et al. filed May 5, 2010, entitled “Methods and Systems for Trans umbilical Breast Augmentation”, the disclosure of which is incorporated in its entirety herein by reference. Depending upon the volume of the excision, reconstruction may be accomplished in a series of post-excision patient visits, in each of which micro volumes of autologous fat are reintroduced until a desired cosmetic result is achieved.

In one example of the present invention, the patient is prepared for either a lumpectomy (when removing cancer from a patient's breast) or a core excision of the parenchyma, or glandular tissue, using generally standard patient preparations known well to those in the art. Specifically, anesthesia may be delivered, as necessary, which is well known in the art. The surgeon may then perform the operation once the patient has been properly prepared for surgery.

When performing a core excision of the parenchyma, the surgeon is removing as much of the glandular tissue as possible. Just as in a mastectomy, the patient is advised that small amounts of breast tissue may be left behind in spite of all efforts. This may apply to all breast tissue on a cellular level. While the method of the present invention described herein is significantly reducing a patient's risk of developing breast cancer, if even a few cells remain after the operation, there may still be a potential risk at developing breast cancer. Therefore, it is important for the surgeon to perform a thorough job at removing as much breast tissue as possible. Improved methods at completely removing all of the breast tissue have been contemplated and any improvements at doing so are within the scope of this invention.

The patient's parenchyma is excised using a variety of methods. Any method involving the excision of the patient's parenchyma may be used to conduct this step of the current invention without departing from its scope. Preferably, the excision of the parenchyma should be conducted such that a minimal amount of scarring and damage is done to the esthetics of the breast. By way of example only, placing an incision around at least a portion of the circumference of the areola may result in a more discrete scar than other areas of the breast. By way of another example, the surgeon may elect to expose the breast tissue by way of an infra-mammary incision, an incision underneath the breast. Any number of approaches for exposing and removing the breast tissue for removal may be incorporated herein without departing from the scope of this invention. The excision of the parenchyma is similar in nature to a lumpectomy with the breast skin, nipple, fat, and surrounding fibrous architecture left unharmed. Therefore, after the excision of the parenchyma, the majority of the breast remains and is left generally intact.

Preferably, the fat used for reconstruction was previously harvested to optimize properties of younger stem cells. By way of example, a woman could have fat cells extracted from her body in her younger years (20's and 30's) and have them cryogenically stored until she no longer needed her ductal tissue (which is required for breast feeding). When the woman no longer needed her ductal tissue (e.g. after breast feeding her youngest child) she could have her ductal tissue removed and replaced with the cryogenically stored fat. This would ultimately eliminate or minimize her potential for the common type of breast cancer associated with the ductal tissue.

It will be appreciated by those skilled in the art and others that all of the functions described in this disclosure may be embodied in software executed by one or more processors of the disclosed components and mobile communication devices. The software may be persistently stored in any type of non-volatile storage.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art. It will further be appreciated that the data and/or components described above may be stored on a computer-readable medium and loaded into memory of the computing device using a drive mechanism associated with a computer readable storing the computer executable components such as a CD-ROM, DVD-ROM, or network interface further, the component and/or data can be included in a single device or distributed in any manner. Accordingly, general purpose computing devices may be configured to implement the processes, algorithms and methodology of the present disclosure with the processing and/or execution of the various data and/or components described above.

Generally described, aspects of the present disclosure relate to the utilization of stored fat cells. Specifically, in one embodiment, fat cells removed from a patient may be subdivided into a number of fat cell subdivisions (e.g. containers) for utilization in a series of medical procedures. The fat cell subdivisions can be prepared for short term storage for use in medical procedures to be performed within a defined period time. Additionally, some portion of the fat cell subdivisions can be prepared for long term storage for use in one or more medical procedures to be performed in the future. Both the short term and long term stored fat cell subdivisions can be further associated with various user information for ensuring the appropriate matching of stored fat cell subdivisions in medical procedures to be performed. In one embodiment, fat cell subdivision storage service providers can provide user (e.g., patients) and medical practitioners with the storage services.

As described above, medical practitioners can also utilize stored fat cell subdivisions (either long term or short term) in a series of procedures on a patient. In one embodiment, a medical practitioner may need to determine the number of stored fat cell subdivision required for a planned procedure and request stored fat cell subdivisions from a fat cell service provider. In turn, the fat cell service provider processes the request and provides the medical practitioner with the requested fat cell subdivisions.

Although various aspects of the disclosure will be described with regard to illustrative examples and embodiments, one skilled in the art will appreciate that the disclosed embodiments and examples should not be construed as limiting. For example, the present disclosure may be described with regard to specific medical procedures for the extraction of tissue and the utilization of extracted fat cells in subsequent procedures. Likewise, although the present application will be discussed with regard to illustrative storage services provided by fat cell subdivision storage service provider, the one skilled in the relevant art will appreciate that the service need not provide all the functionality that may be otherwise attributed to the fat cell subdivision storage service provider or that some portion of the functionality attributed to a fat cell subdivision storage service provider may be provided by the user or the medical practitioner.

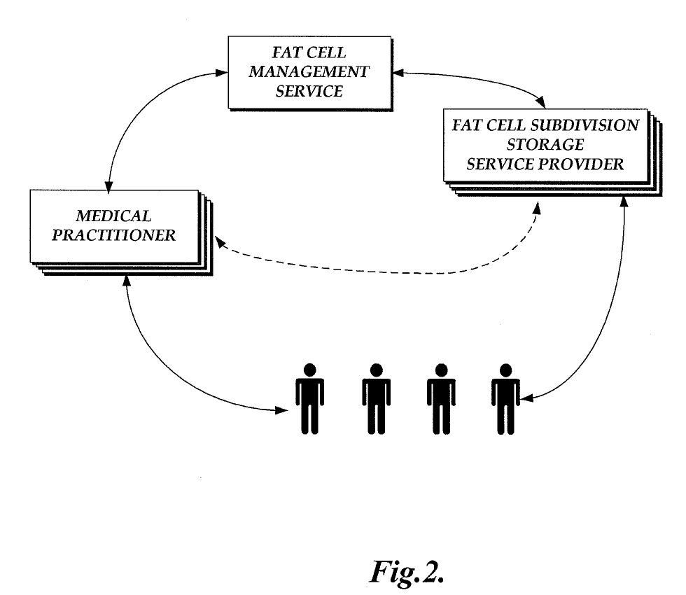

With reference to FIG. 2, a block diagram illustrative of fat cell subdivision utilization environment including a number of users (e.g., patients or system administrators), a set of medical practitioners, a fat cell subdivision management service, and a set of fat cell subdivision storage providers is provided. Although the fat cell management service, fat cell subdivision storage service provider and medical practitioner are illustrated as separate components, one skilled in the relevant art will appreciate that various combinations of components may occur. Additionally, the illustrative actions/processes attributed to one particular component in the fat cell subdivision utilization environment may be performed by another component or distributed in a manner to be performed by multiple components of the fat cell subdivision utilization environment.

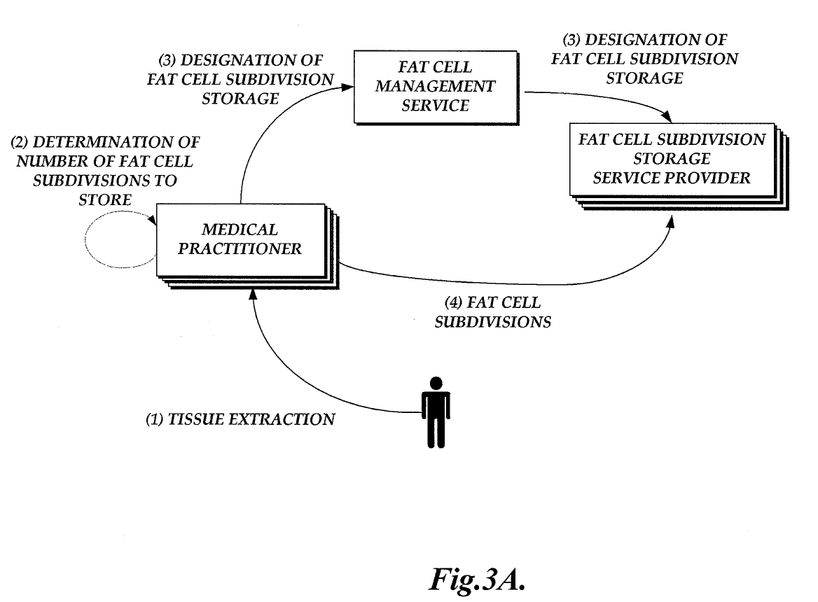

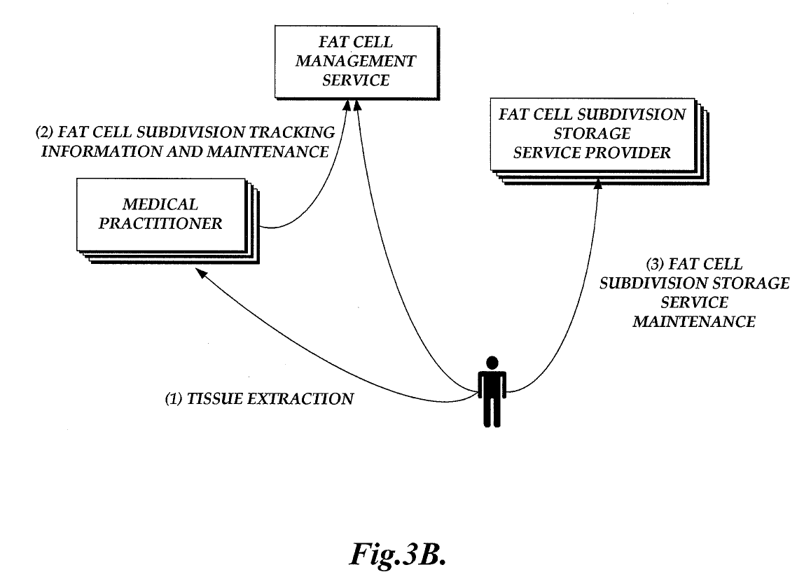

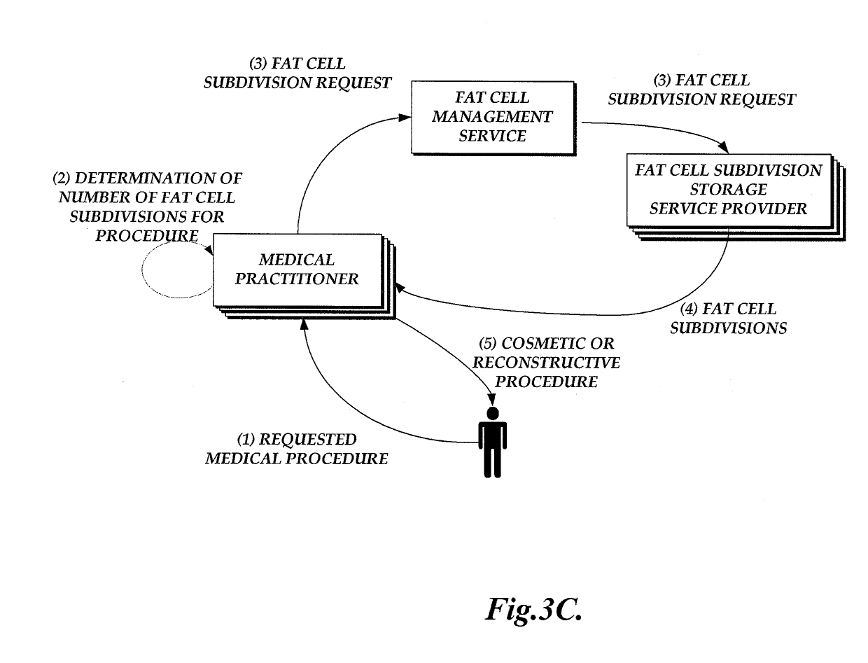

With reference to FIGS. 3A-3C, an illustrative interaction between the component of the fat cell subdivision utilization environment are illustrated including the extraction of tissue, creation and storage of fat cell subdivisions, maintenance of stored fat cell subdivisions and utilization of fat cell subdivisions in subsequent procedures. One skilled in the relevant art will appreciate, however, that the illustrated interaction is only illustrative in natures and that a number of variations and alternative interactions may be encompassed and are within the scope of the present disclosure.

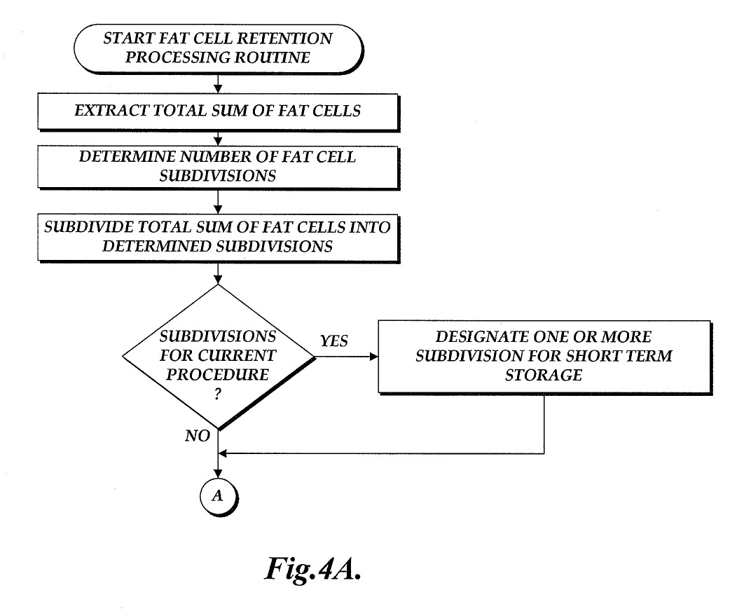

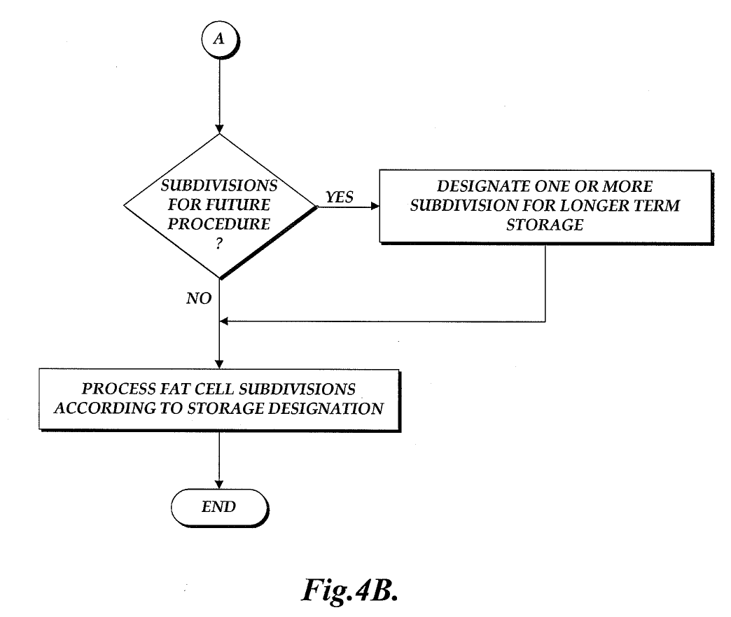

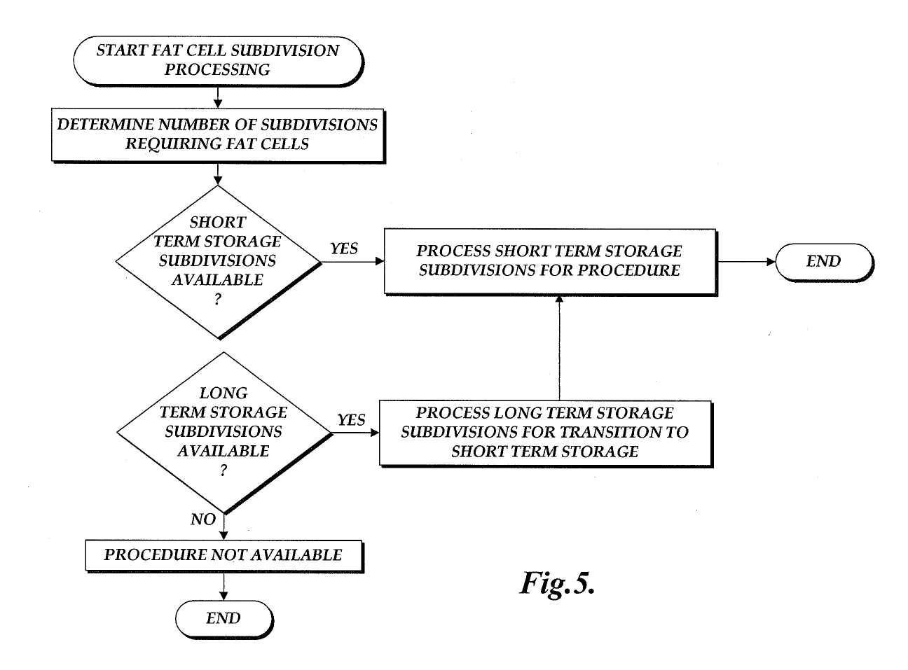

FIGS. 4A, 4B and 5 are flow diagrams illustrative of various procedures for use in the creation, storage and utilization of fat cell subdivisions. The procedures illustrated in FIGS. 4 and 5 may be implemented by a fat cell managements service, medical practitioner, fat cell subdivision storage service provider. For purposes of illustration, FIGS. 4 and 5 can be considered to be implemented by a medical practitioner. However, as previously mentioned, however, attributed to one particular component in the fat cell subdivision utilization environment may be performed by another component or distributed in a manner to be performed by multiple components of the fat cell subdivision utilization environment.

In conjunction with the flow diagrams, the below description illustrates various procedures and functionality that may be performed in the fat cell subdivision utilization environment.

It should be emphasized that many variations and modifications may be made to the above-described embodiments, the elements of which are to be understood as being among other acceptable examples. All such modifications and variations are intended to be included herein within the scope of this disclosure and protected by the following claims.

1. A method for managing tissue removal comprising:
determining a number of fat cell subdivisions for storing fat cells removed from a patient,
designating a storage designation for the number of fat cell subdivisions, wherein the designated storage designation corresponds to at least of one of long term storage and short term storage;
obtaining a designation of a procedure utilizing one or more stored fat cell subdivisions;
determining a number of fat cell subdivisions to be utilized in the procedure; and
causing the preparation of previously stored fat cell subdivisions based on the number of determined fat cell subdivisions, wherein the previously stored fat cell subdivisions may be fat cell subdivisions stored in at least one of short term storage and long term storage.

2. A method of managing adipose tissue, comprising the steps of:
performing liposuction on a patient, to harvest a volume of adipose tissue;
dividing the volume of tissue into at least two containers; and
cryopreserving the containers.

3. The method of claim 2, wherein a first container has a first volume and a second container has a second volume.

4. The method of claim 3, wherein a first set of at least two containers each has a first volume and a second set of at least two containers has a second volume.

5. The method of claim 3, further comprising the step of placing at least the first container into a sterile enclosure prior to the cryopreserving step.

6. The method of claim 2 further comprising the step of associating patient identifying information with each container.

7. The method of claim 2, wherein at least a first container is stored at a first location for reinjection into the patient, and at least a second container is sent to a long term cryogenic storage facility.

8. A method of performing a cosmetic procedure on a patient, comprising the steps of:
harvesting adipose tissue from the patient;
processing the adipose tissue;
dividing the adipose tissue into at least two sets;
reinjecting at least a portion of a first set back into the patient; and
sending the second set to a long term cryogenic storage facility.

9. A method of performing a cosmetic procedure, comprising the steps of:
obtaining a volume of adipose tissue from cryogenic storage, the tissue contained in a sterile container which is enclosed by a nonsterile container;
removing the nonsterile container outside of a sterile field;
introducing the sterile container into the sterile field; and
opening the sterile container to expose the adipose tissue within the sterile field.

10. The method according to claim 9, further comprising attaching the sterile container to an injection device, and administering said adipose tissue to a target region of a subject via said injection device.

11. The method according to claim 10, wherein said injection device comprises a pump that controls the volume of adipose administered.

12. The method according to claim 11, wherein said pump is configured to administer said adipose tissue in a plurality of unit volumes, each unit volume within the range of from about 0.1 mL to about 1.0 mL per administration.
